(19) United States
(12) Patent Application Publication
(10) Pub. No.: US 2011/0031329 A1
(43) Pub. Date: Feb. 10, 2011

(54) VAPOR TRANSFER HAND PIECE

(76) Inventor: Bertram Balch, Torrance, CA (US)

Correspondence Address: KLEIN, O'NEILL & SINGH, LLP, 18200 VON KARMAN AVENUE, SUITE 725, IRVINE, CA, 92612, US

(21) Appl. No.: 12/853,033

(22) Filed: Aug. 9, 2010

Related U.S. Application Data
(60) Provisional application No. 61/232,366, filed on Aug. 7, 2009.

(51) Int. Cl. A61L 9/03; B23P 17/04

(52) U.S. Cl. 239/34; 29/428

(57) ABSTRACT

Vapor transfer devices are generally discussed herein with particular discussions extended to a vapor transfer hand piece configured to work with a thermal vaporizer. The vapor transfer hand piece provided according to aspects of the present invention comprises a tip made from a first material, which is connected with a handle made from a second material through a coupling made from a third material, wherein the thermal conductivity of the third material is lower than that of the first material such that the heat transfer from the tip to the handle is insulated by the coupling.

### CROSS-REFERENCE TO RELATED APPLICATION

This application claims the benefit of U.S. provisional Application No. 61/232,366, filed Aug. 7, 2009, the contents of which in their entireties are hereby incorporated by reference.

### FIELD OF ART

Vapor transfer devices are generally discussed herein with particular discussions extended to vapor transfer hand pieces configured for use with thermal vaporization devices.

### BACKGROUND

Vaporizing carriers for aroma-therapy such as medicine herbs and flower essences to release active elements and essences are well known in the art. In general, the vaporization process includes directly heating a carrier in a pipe and then inhaling the smoke released from the heated carrier. As used herein, a carrier is a substance, such as herbs, tobacco, dried flowers, etc., that contain moisture and/or oil that can be vaporized.

Vaporizer devices for vaporizing carriers, such as the ones manufactured by the inventor of the present invention, are well known and are disclosed in U.S. Pat. Nos. 7,445,007 and 7,475,684, the contents of which are incorporated herein by reference. An exemplary prior art vaporizer apparatus 10 is shown in FIG. 1A, which comprises among other things a housing 12 comprising in part a heating element assembly 16, a power regulator 20 and a shield 30. The vaporizer apparatus is used with a hand piece 22 by engaging the hand piece to the shield 30 and then holding the hand piece in the engaged position during inhalation so that air may transfer from the vaporizer apparatus 10 through the hand piece and then to the user. The hand piece 22 is optionally connected to a tubing 32 for inhaling at a spaced or remote location from the end of the hand piece. A screen 36 is fitted within the hand piece 22 to capture or retain the carrier within the hand piece. As hot air drawn by the user passes through the carrier in the hand piece, oil essences from the carrier are vaporized for inhaling by the user.

The vapor transfer hand piece of the present invention improves the overall use and experience of using a vaporizer to vaporize essences from a carrier.

### SUMMARY

The present invention may be practiced by providing a vapor transfer hand piece comprising a tip made of a first material attached to a handle made from a second material through a coupling made from a third material; the coupling comprises two open slip-on ends each comprising an internal shoulder; wherein the tip and the handle are attached to a respective slip-on end of the coupling; and wherein the first material has a first heat conductivity value, which is higher than the heat conductivity value of the third material so that heat conducting through the tip is insulated by the coupling from conducting to the handle.

In yet another aspect of the present invention, there is provided a vapor transfer hand piece comprising: a coupling connecting a tip to a handle; a screen disposed within the coupling; wherein the coupling is made from a material with a thermal conductivity lower than that of the tip so that the heat transfer from the tip to the handle is insulated by the coupling.

In yet another aspect of the present invention, there is provided a method for forming a vapor transfer hand piece, the method comprising providing a tip made of a first material; providing a handle made from a second material; providing a coupling made from a third material; the coupling comprises two open slip-on ends each comprising an internal shoulder; and attaching the tip and the handle to a respective slip-on end of the coupling; wherein the first material has a first heat conductivity value, which is higher than the heat conductivity value of the third material so that heat conducting through the tip is insulated by the coupling from conducting to the handle.

Other aspects and variations of the vapor transfer hand piece summarized above are also contemplated and will be more fully understood when considered with respect to the following disclosure.

### BRIEF DESCRIPTION OF THE DRAWINGS

These and other features and advantages of the present invention will become appreciated as the same become better understood with reference to the specification, claims and appended drawings wherein:

FIG. 1 is a semi-schematic cross-sectional view of a prior art thermal vaporizer apparatus;

FIG. 2 is a side view of a vapor transfer hand piece provided in accordance with aspects of the present invention shown in an assembled state;

FIG. 3 is an exploded side view of the vapor transfer hand piece of FIG. 2;

FIG. 4 is an illustration of a cross-sectional side view of the coupling shown in FIG. 3;

FIG. 5 is a cross-sectional side view of the vapor transfer hand piece of FIG. 2; and

FIG. 6 is a cross-sectional side view of an alternative tip provided in accordance with aspects of the present invention.

### DETAILED DESCRIPTION

The detailed description set forth below in connection with the appended drawings is intended as a description of the presently preferred embodiments of the vapor transfer hand piece for use with a vaporizer in accordance with aspects of the present invention and is not intended to represent the only forms in which the present invention may be constructed or utilized. The description sets forth the features and the steps for constructing and using the hand piece of the present invention in connection with the illustrated embodiments. It is to be understood, however, that the same or equivalent functions and structures may be accomplished by different embodiments that are also intended to be encompassed within the spirit and scope of the invention. As denoted elsewhere herein, like element numbers are intended to indicate like or similar elements or features

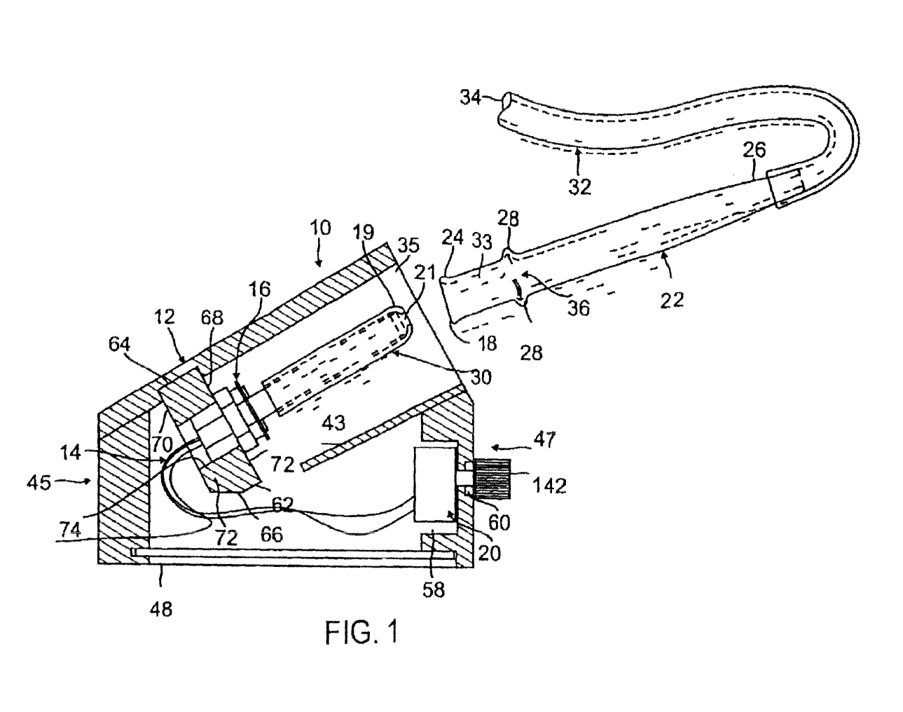

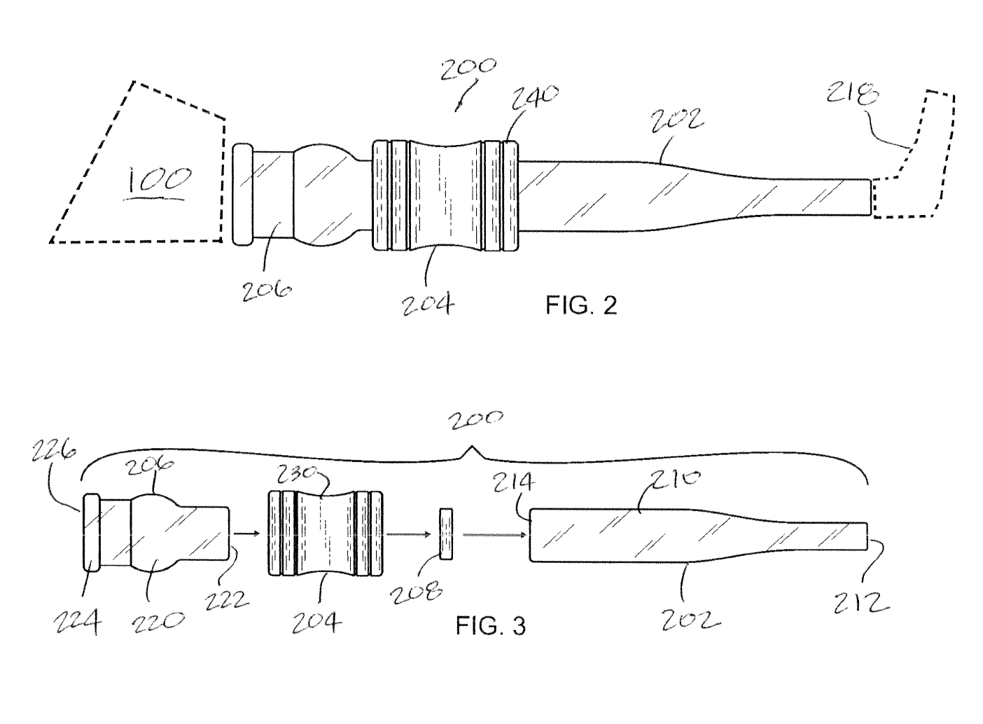

FIG. 2 is a side view of an embodiment of a vapor transfer hand piece 200 shown in an assembled state provided in accordance with aspects of the present invention. In one exemplary embodiment, the hand piece 200 is configured for use with a thermal vaporizer apparatus, such as the one shown in FIG. 1. As shown in FIG. 2, the hand piece 200 comprises a handle 202 connected to a tip 206 via a coupling, 204. The handle 202 normally connects to a flexible tubing 218, shown in phantom and resembles tubing 32 in FIG. 1, leading to a draw end for inhalation by a user. However, optionally the handle 202 may be used directly without the flexible tubing 218 by inhaling directly from the user inhale end 212, also referred to as an outlet end. A screen 208 (FIG. 5) is disposed within the coupling 204. As further discussed below, the screen 208 is configured to retain the carrier within the space upstream of the screen, which is packed or placed therein by a user prior to using the hand piece to vaporize the essences. In use, the tip 206 is connected to or mated against a glass shield that is mounted over a heating element of a vaporizer 100, which is shown in phantom in FIG. 2 and can be any prior art vaporizers, such as the one shown in FIG. 1. In one embodiment, the coupling 204 and the handle 202 are integrally or unitarily formed from a single material, such as from a silicone material or engineered plastic.

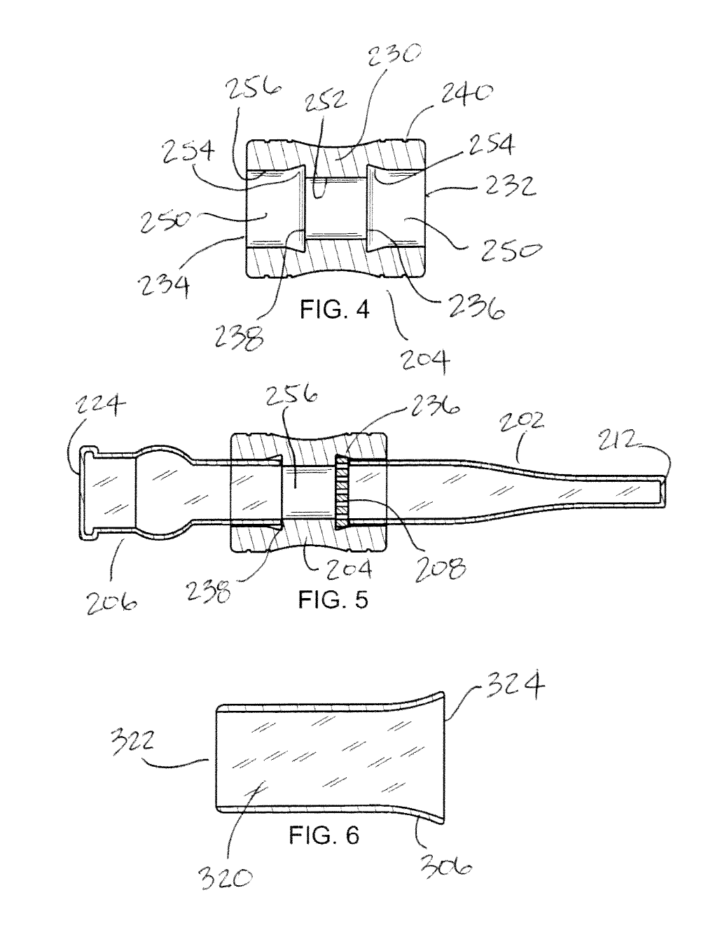

FIG. 3 illustrates an exploded side view of the hand piece 200 of FIG. 2. FIG. 4 shows a cross-sectional side view of the coupling 204 shown in FIGS. 2 and 3. FIG. 5 is a cross-sectional side view of FIG. 2. As set forth above, the hand piece 200 comprises the handle 202, the coupling 204, the screen 208 and the tip 206, which are further discussed below. In other embodiments, the hand piece comprises more than four components, such as with an additional screen or a coupling made from two or more assembled components.

In one embodiment, the handle 202 is made from a transparent or opaque glass. In other embodiments, the handle is made from other rigid or semi-rigid materials such as engineered plastic, PFA, PEEK, PTFE, Vespel polyimide, ceramic etc. The material selection 202 of the handle determines its thermal conductivity value. Thus, for example, a handle made of glass has a thermal conductivity value k of 1.05 W/mK at 25° C., where W is the amount of heat transfer per unit area in meter per unit temperature in Kelvin. In contrast, at the same temperature, the thermal conductivity of a handle made of aluminum is 250 W/mK, of brass is 109 W/mK, and or of iron is 80 W/mK (See, e.g., Table 1 for thermal conductivity values of various materials). Likewise, material selection of the coupling and of the tip determines each component's thermal conductivity value.

In an alternative embodiment, the coupling is made from two or more materials, such as from an outer silicone, rubber, or other plastic material layer and an inner layer, such as from a metal layer. As non-limiting examples, the inner layer may be made from aluminum, copper, or Fe203 iron oxide or alloys thereof. The multi-layer allows the coupling to remain cool to the touch but allow for some transfer of conductivity between the tip and the handle. In short, surface temperatures of the tip, of the coupling, and/or of the handle may be regulated or controlled through material selection or by incorporating multiple layers and materials.

As alluded to above, the vapor transfer hand piece 200 is used by holding the tip 206 against a glass shield, which is hot due to being placed over a heating element. Thus, heat transfer normally flows through the center of the hand piece in the form of convection, but also through the hand piece itself due to conduction. In one embodiment, the entire hand piece 200 is made from one or more materials having low thermal conductivity values to minimize conduction through the hand piece, which may be touched by the user. Thus, looking at the table, plastic is preferred to minimize conduction. However, glass is a more preferred material because in addition to being inert and non reactive to the vapor generated during the vaporization process, it possesses a relatively low thermal conductivity compared to various metals summarized in the table and it is more aesthetically appealing.

TABLE 1
Thermal conductivities of exemplary common materials at 25° C.:

| | Thermal Conductivity, |
|---|---|
| MATERIAL | k (W/mK) |
| Aluminum | 250 |
| Brass | 109 |
| Iron | 80 |
| Glass | 1.05 |
| Insulation materials | 0.035-0.16 |
| Plastic, foamed | 0.03 |
| Silicone rubber | 0.22 |
| Butadiene Rubber | 0.25 |
| Cloroprene Rubber | 0.18 |
| Natural Rubber | 0.13 |
| Syrenebutadiene Rubber | 0.2-0.25 |
| Urethane Rubber | 0.16 |
| Urethane Foam | 0.021 |
| Wood, oak | 0.17 |

As shown in FIGS. 3 and 5, the handle 202 comprises a handle body 210 with two open ends. In one preferred embodiment, the handle body 210 has a tapered cylindrical shape with one end bigger than the other end; however, other appropriately shaped tube configurations are also suitable. At one end, the handle body 210 comprises an outlet end 212 for connecting to any flexible tubing or extension member 218 (FIG. 2 in phantom). The outlet end 212 is preferably tapered along the longitudinal axis of the handle body 210 to a dimension suitable for a slip on fit with commercially available tubing or extension members, such as tubing 32 shown in FIG. 1. In other embodiments, the outlet end 212 is configured to receive or accommodate a tubing as an inside fit. At the other end, the handle body 210 comprises a linking end 214 having a generally straight cylindrical end configured for a slip on fit with the coupling 204. In one embodiment, the linking end 214 has a raised surface or lip (not shown), which resembles a bead or ring rising from a generally constant outer surface, for engaging the coupling 204 in an interference fit. For example, the coupling 204 can have a first opening 232 of a first internal diameter and the raised surface or lip has a slightly larger outside diameter dimension so that when the linking end 214 is pushed into the opening 232, it results in an interference fit. In alternative embodiments, instead of a raised surface, the linking end 214 is smooth and dimensioned to engage in a friction fit with the first opening 232 of the coupling 204. Even with a friction fit, it is understood that some nominal interference is possible.

The tip 206 connects the hand piece 200 to a vaporizer apparatus 100 (shown in phantom in FIG. 2 and can embody the device 10 of FIG. 1). The tip 206 may be made from a transparent or opaque glass or other rigid or semi-rigid material. The thermal conductivity value of the tip 206 depends on the material from which it is made. As the tip 206 is configured to connect directly to a vaporizer, the heat transfer from a heating element of the vaporizer apparatus to the tip 206 can render it hot to the touch. The higher the thermal conductivity property of the tip 206, the hotter the tip 206 gets during the vaporization process. Thus, in a preferred embodiment, the tip 206 is made from glass, which in addition to being inert and non-reactive to the shield and hot vapor generated during the vaporization process, has a relatively low thermal conductivity (1.05 W/mK) compared to metal, for example. However, the tip may be made from other materials with relatively low thermal conductivities, such as engineered plastic and ceramic.

As shown in FIGS. 3 and 5, the tip 206 comprises a tip body 220 having two open ends. In one preferred embodiment, the tip body 220 has a cylindrical shape comprising sections of various diameters; however, other appropriately shaped tube configurations are also suitable. At one end, the tip 206 comprises a coupling end 222 for a slip on fit with the coupling 204. In one embodiment, the coupling end 222 has a raised surface or lip for engaging in an interference fit with the coupling 204. For an interference coupling, the coupling end 224 is dimensioned such that its diameter is relatively larger than an inside diameter of the second opening 234 of the coupling 204. In alternative embodiments, instead of a raised surface, the coupling end 224 is smooth and dimensioned to engage in a friction fitting with the second opening 234 of the coupling 204. In yet another embodiment, the coupling end 224, having evenly enlarged diameter throughout the entire section of the coupling end, has an interference fit with the second opening 234 of the coupling.

At the opposite end, the tip body 220 comprises an inlet end 224 for connecting to a vaporizer apparatus (FIG. 1), as previously discussed. In one embodiment, the inlet end 224 includes a flared or tapered section 226 that is dimensioned to slip on snuggly over a dome 19 of the glass shield 30 located on the heating element of the vaporizer apparatus shown in FIG. 1. The fit between the tapered section 226 and the dome 19 on the glass shield 30 is such that the entire hand piece is secured and to the vaporizer without external support, such as with a user's hand. This fit, which is known as a “hand free” hand piece, does not require the end user to hold the hand piece against the vaporizer apparatus during use. In other embodiments, the hand piece needs to be held in the contacted position by the end user, as further discussed below.

As set forth above, the coupling 204 connects the handle 202 to the tip 206. In the embodiment shown, the coupling 204 comprises a coupling body 230, which can be made from any suitable natural, synthetic, composite, and laminate materials, including, without limitation, silicone elastomer, rubber, plastic, and/or combinations thereof. Preferably, the thermal conductivity value of the coupling body 230 is lower than the thermal conductivity value of the tip 206 so that heat generated during the vaporization process and transferred to the tip 206 during use is insulated from conducting through to the handle 202. In a preferred embodiment, the coupling 204 is made from materials with low thermal conductivities to act as an insulator against heating the handle 202 through conduction heating. In one embodiment, the coupling 204 is made from silicone rubber, which in addition to being relatively non-reactive and inert to the vapor generated during the vaporization process, has a thermal conductivity in the range of 0.2 W/mK. In alternative embodiments, other non-reactive and inert elastomers with low thermal conductivities may also be used. Non-limiting examples of suitable elastomers with low thermal conductivities are provided in Table 1. Non-reactive materials with thermal conductivities lower than the value of glass are preferred. In a specific embodiment, the coupling is made from a pliable material having a thermal conductivity value of less than 1 W/mK. Still in another embodiment, the conductivity values of the tip, the coupling, and the handle are within the order of three or less from one another thus making them substantially on par with a singularly formed handle, which is the same from end to end.

The coupling body 230 comprises two open ends, corresponding to the first opening 232 and the second opening 234, which are configured to engage the handle 202 and the tip 206, respectively. In one embodiment, the coupling body 230 has a cylindrical shape with two slightly flared exterior ends that resembles an hourly-glass exterior figure. Internally, the two bores 250 have interior surfaces that are slightly tapered in the direction from near the center of the coupling to the two open ends. However, in alternative embodiments, the two bores 250 may have the same or somewhat constant inside diameter throughout to form a snap fit with the coupling and the handle. In yet other embodiments, the coupling body 230 may have other appropriately shaped bore configurations, such as helical or thread shape. In still yet another alternative embodiment, the coupling incorporates features of the tip so that the hand piece is made from a coupling and a handle only, without a separate tip. In this variation, the coupling may take on a ground tapered connection for mating with the shield on the vaporizer apparatus.

FIG. 4 shows a cross-sectional view of the coupling 204. As illustrated, the coupling has a reduced internal section 252 located adjacent the two bores to define a first internal shoulder 236 and second internal shoulder 238. The screen 208 may be seated against the first shoulder 236 or the second shoulder 238 (FIG. 5). Although shown seated adjacent the first shoulder 236 (FIG. 5), the screen is preferably seated in the same bore as the tip 206, i.e., the second shoulder 238 in FIG. 5. In one embodiment, the screen is made from ceramic material and resembles a honey-cone disc. In another embodiment, the screen is made from metal and resembles a mesh, which serves to retain carriers or solid materials loaded in the chamber upstream of the screen (i.e., to the left of the screen in FIG. 5) for vaporizing by the user during the vaporization process.

In one embodiment, the two shoulders 236, 238 each incorporates a recessed groove 254, which corresponds to a larger internal diameter than the internal diameter of the bore closer to the inlet, such as at point 256. The recessed grooves 254 are configured to accommodate a screen and/or a bead located on a connection end of either the handle 202 or the tip 206.

The internal shoulders 236 and 238 limit how far the handle 202 and the tip 206 fit into the coupling body 230. The coupling also crushes down during assembly and spring back slightly to firmly hold the tip and the handle. Thus, in addition to the easy slip on fitting, no alignment of the components is required for the assembly of the hand piece 200. Indeed, to assemble the hand piece 200, regardless whether the engagement is friction or interference fit, the linking end 214 of the handle 202 and the connection end 222 of the tip 206 are pushed into the first opening 232 and the second opening 234, respectively, until they abut the first internal shoulder 236 and the second internal shoulder 238, respectively. In one exemplary embodiment, the coupling body 230 is symmetrical such that the first opening 232 and second opening 234 are identical to each other, and the first internal shoulder 236 and second internal shoulder 238 are identical to each other as well as are equally spaced. In this preferred embodiment, the handle 202 and the tip 206 are connectable to the coupling 204 via either opening. Similarly, the screen 208 is seatable within either the first internal shoulder 236 or second shoulder 238. In alternative embodiments, however, the coupling body 230 is asymmetrical and the handle 202 and the tip 206 are connectable only to a respective opening on the coupling 204. The simple connect/disconnect capabilities of the vapor transfer hand piece allows for easy cleaning of the load chamber 256. For example, the tip and the handle are readily removable from the coupling by moving them axially away from the coupling so that the inside of the coupling as well as the screen may be cleaned.

As shown in FIG. 4, tapered exterior grooves 240 may be added to enhance aesthetic appeal of the hand piece. To further facilitate assembly, the hand piece 200 may incorporate one or more labels or logos 228 on their respective exterior wall surfaces, such as by selectively painting, on a label, message, or note at the inlet end 224 of the tip 206 to distinguish it from the coupling end 222. Alternatively or in addition thereto, a warning label may be added to warn the end user that it may be hot to the touch. Alternatively, labels or logos 228 may be added to other components of the hand piece, such as onto the handle and/or to the coupling, either for instruction purposes or to enhance its aesthetic appeal.

FIG. 6 shows an alternative tip 306 of a vapor transfer hand piece provided in accordance with aspects of the present invention. Similar to previously described embodiments, the tip 306 is configured to connect to a handle 202 via a coupling 204 with a screen 208 disposed therein, such as that shown in FIG. 5 but with the tip of FIG. 6. A hand piece having the tip of FIG. 6 may be referred to as a “handheld” version as the inlet end 324 is configured to merely abut a glass shield 30 of a vaporizer but not couple to the glass shield and therefore requires external support, such as support from a user's hand. In one embodiment, the tip 306 comprises a coupling end 322 configured to fit with a coupling 204 in a slip on fit. Similar to the previously described embodiment, the coupling end 322 can engage the coupling 204 via an interference fit or friction fit. In alternative embodiments, the fit is threaded engagement, which requires matching engageable threads between the coupling and the two connection ends of the tip and the handle.

The various components of the hand piece 200, including but not limited to the handle 202, the coupling 204, the tip 206/306 and the screen 208 provided according to aspects of the present invention, may be conventionally produced. As set forth above, the tip 206/306 and the handle 202 are made from inert and non-reactive materials with relatively low thermal conductivity properties. In one embodiment, the tip 206/306 and the handle 202 are both made from glass and may be produced by glass blowing or alternative methods that are well known in the art. The coupling 204 preferably is made from an inert elastomer, with a thermal conductivity value lower than that of the tip 206/306 and/or the handle 202 In a specific embodiment, the coupling has a thermal conductivity value lower than 1 W/mK. In a specific embodiment, the coupling 204/304 is made of silicone rubber and may be made by injection molding methods which are well known in the art. In alternative embodiments, wherein other inert and non-reactive elastomers with low thermal conductivities are used, well known injection molding methods may be used.

Thus, aspects of the present invention is understood to include a vapor transfer hand piece for use with a vaporizer, said hand piece comprising a tip axially insertable into a coupling and a tip axially insertable into the coupling. In a specific embodiment, the coupling as a thermal conductivity value that is less than the thermal conductivity value of either the tip or the handle. In a preferred embodiment, the value is at least two times less.

A further aspect of the present invention is understood to include a method for cleaning a vapor transfer hand piece, said method comprising axially moving a tip away from a coupling, axially moving a handle away from the coupling, axially moving a screen away from the coupling, and removing spent carriers from inside the coupling.

Many alterations and modifications may be made by those having ordinary skill in the art, without departing from the spirit and scope of the invention, Therefore, it must be understood that the illustrated embodiments have been set forth only for the purposes of examples, and that the embodiments should not be taken as limiting the invention as defined by the following claims. The following claims are, therefore, to be read to include not only the combination of elements which are literally set forth, but all equivalent elements for performing substantially the same function in substantially the same way to obtain substantially the same result. The claims are thus to be understood to include those that have been illustrated and described above, those that are conceptually equivalent, and those that incorporate the ideas of the invention.

What is claimed is:

1. A vapor transfer hand piece comprising:
a tip made of a first material attached to a handle made from a second material through a coupling made from a third material;
the coupling comprises two open slip-on ends each comprising an internal shoulder;
wherein the tip and the handle are attached to a respective slip-on end of the coupling; and
wherein the first material has a first heat conductivity value, which is higher than the heat conductivity value of the third material so that heat conducting through the tip is insulated by the coupling from conducting to the handle.

2. The vapor transfer hand piece of claim 1, wherein the first material is the same as the second material.

3. The vapor transfer hand piece of claim 1, wherein the first material is glass.

4. The vapor transfer hand piece of claim 1, wherein the third material is silicone.

5. The vapor transfer hand piece of claim 1, further comprising a screen made from a fourth material disposed inside the coupling.

6. The vapor transfer hand piece of claim 5, wherein the fourth material is ceramic.

7. The vapor transfer hand piece of claim 1, wherein at least one of the tip and the handle has a raised lip.

8. The vapor transfer hand piece of claim 1, wherein the coupling is symmetrical.

9. A vapor transfer hand piece comprising:
a coupling connecting a tip to a handle; a screen disposed within the coupling;
wherein the coupling is made from a material with a thermal conductivity lower than that of the tip so that the heat transfer from the tip to the handle is insulated by the coupling.

10. The vapor transfer hand piece of claim 9, wherein the coupling is made of silicone.

11. The vapor transfer hand piece of claim 9, wherein the thermal conductivity of the coupling is less than 1 W/mK.

12. The vapor transfer hand piece of claim 9, wherein the screen is made of ceramics.

13. The vapor transfer hand piece of claim 9, wherein the tip is made of glass.

14. The vapor transfer hand piece of claim 9, wherein the tip and the handle are connected to the coupling by slip on fitting.

15. A method of forming a vapor transfer hand piece comprising:
providing a tip made of a first material;
providing a handle made from a second material;
providing a coupling made from a third material; the coupling comprises two open slip-on ends each comprising an internal shoulder; and
attaching the tip and the handle to a respective slip-on end of the coupling;
wherein the first material has a first heat conductivity value, which is higher than the heat conductivity value of the third material so that heat conducting through the tip is insulated by the coupling from conducting to the handle.

16. The method of claim 15, wherein the first material is the same as the second material.

17. The method of claim 15, wherein the first material is glass.

18. The method of claim 15, wherein the third material is silicone.

19. The method of claim 15, further providing a screen made from a fourth material disposed inside the coupling.

20. The method of claim 19, wherein the fourth material is ceramic.

21. The method of claim 15, wherein at least one of the tip and the handle has a raised lip.
